(19) United States
(12) Patent Application Publication
(10) Pub. No.: US 2025/0213380 A1
(43) Pub. Date: Jul. 3, 2025

(54) POSTURE IMPROVEMENT ASSISTANCE SYSTEM, PROCESSING DEVICE, POSTURE IMPROVEMENT ASSISTANCE METHOD, AND PROCESSING METHOD

(71) Applicant: Mitsubishi Electric Corporation, Tokyo (JP)

(72) Inventor: Masami HAGIWARA, Tokyo (JP)

(73) Assignee: Mitsubishi Electric Corporation, Tokyo (JP)

(21) Appl. No.: 19/086,151

(22) Filed: Mar. 21, 2025

Related U.S. Application Data
(63) Continuation of application No. PCT/JP2022/036627, filed on Sep. 30, 2022.

(51) Int. Cl. A61F 5/02; A61B 5/11

(52) U.S. Cl. CPC A61F 5/02; A61B 5/11

(57) ABSTRACT

A posture improvement assistance system includes: an input device that receives input of user information which is information regarding a posture of a user; a processing device that generates a current silhouette representing a current posture of the user on the basis of the user information; and an output device that outputs the current silhouette and an ideal silhouette representing an ideal posture of the user.

### CROSS-REFERENCE TO RELATED APPLICATION

This is a continuation application of PCT/JP2022/036627, filed on Sep. 30, 2022, the entire contents of which are incorporated herein by reference in their entirety.

### TECHNICAL FIELD

The present disclosure relates to a posture improvement assistance system, a processing device, a posture improvement assistance method, and a processing method.

### BACKGROUND ART

An object of a walking mode display system described in Patent Literature 1, which is common to the posture improvement assistance system according to the present disclosure in improving a posture of a pedestrian or the like, is to analyze a walking mode of a pedestrian, to display the walking mode of the pedestrian in an easy-to-understand manner, and to present an improvement method. In order to achieve the object, the walking mode display system describes feature amounts calculated at measurement points indicated by circles in two skeleton models imitating respective bodies of a reference pedestrian and a user.

### CITATION LIST

### Patent Literature

Patent Literature 1: WO 2019-208312 A

### SUMMARY OF INVENTION

### Technical Problem

However, in the walking mode display system, the feature amounts are described with a technical content such as “peak intensity of a cross correlation function of speeds on Z-axis of a left hand and Z-axis of a left thumb”, therefore, a person not having technical knowledge cannot understand the description of the feature amounts, and as a result, it is difficult for the person not having technical knowledge to perform the improvement of the walking mode.

An object of the present disclosure is to provide a posture improvement assistance system, a processing device, a posture improvement assistance method, and a processing method that enable easier improvement of a posture than related art.

### Solution to Problem

In order to solve the problems, the posture improvement assistance system according to the present disclosure includes: processing circuitry configured to receive input of user information which is information regarding a posture of a user; generate a current silhouette representing a current posture of the user on the basis of the user information; and output the current silhouette and an ideal silhouette representing an ideal posture of the user.

### Advantageous Effects of Invention

The posture improvement assistance system according to the present disclosure enables easier improvement of a posture than related art.

### BRIEF DESCRIPTION OF DRAWINGS

FIG. 1 is a functional block diagram of a posture improvement assistance system SKS of a first embodiment.

FIG. 2 illustrates a position of an input device NY of the first embodiment (part 1).

FIG. 3 illustrates a position of the input device NY of the first embodiment (part 2).

FIG. 4 illustrates a position of an output device SY of the first embodiment (part 1).

FIG. 5 illustrates a position of the output device SY of the first embodiment (part 2).

FIG. 6 illustrates an operation of a processing device SH of the first embodiment.

FIG. 7A illustrates a mode (part 1) output by the output device SY of the first embodiment. FIG. 7B illustrates a mode (part 2) output by the output device SY of the first embodiment. FIG. 7C illustrates a mode (part 3) output by the output device SY of the first embodiment.

FIG. 8A illustrates a type (part 1) of the output device SY of the first embodiment. FIG. 8B illustrates a type (part 2) of the output device SY of the first embodiment. FIG. 8C illustrates a type (part 3) of the output device SY of the first embodiment. FIG. 8D illustrates a type (part 4) of the output device SY of the first embodiment. FIG. 8E illustrates a type (part 5) of the output device SY of the first embodiment. FIG. 8F illustrates a type (part 6) of the output device SY of the first embodiment. FIG. 8G illustrates a type (part 7) of the output device SY of the first embodiment.

FIG. 9A illustrates one mode (part 1) of projection of the output device SY of the first embodiment. FIG. 9B illustrates one mode (part 2) of the projection of the output device SY of the first embodiment.

FIG. 10 illustrates alternatives to a current silhouette GS, an ideal silhouette RS, and the like of the first embodiment.

FIG. 11 illustrates a configuration of a database DB of the first embodiment.

FIG. 12 is a functional block diagram of the processing device SH of the first embodiment.

FIG. 13 illustrates a hardware configuration of each of the posture improvement assistance system SKS and the processing device SH of the first embodiment.

FIG. 14 illustrates a hardware configuration based on software implementation of each of the SKS and the processing device SH of the embodiment.

FIG. 15 is a flowchart illustrating an operation of the posture improvement assistance system SKS of the first embodiment.

FIG. 16 is a flowchart illustrating an operation of the processing device SH of the first embodiment.

FIG. 17 illustrates a mode (part 1) of display or the like of the posture improvement assistance system SKS of the first embodiment.

FIG. 18A illustrates a mode (part 2-1) of the display or the like of the posture improvement assistance system SKS of the first embodiment. FIG. 18B illustrates a mode (part 2-2) of the display or the like of the posture improvement assistance system SKS of the first embodiment. FIG. 18C illustrates a mode (part 2-3) of the display or the like of the posture improvement assistance system SKS of the first embodiment. FIG. 18D illustrates a mode (part 2-4) of the display or the like of the posture improvement assistance system SKS of the first embodiment.

FIG. 19A illustrates an operation (part 1-1) of a posture improvement assistance system SKS of a second embodiment. FIG. 19B illustrates an operation (part 1-2) of the posture improvement assistance system SKS of the second embodiment. FIG. 19C illustrates an operation (part 1-3) of the posture improvement assistance system SKS of the second embodiment.

FIG. 20A illustrates an operation (part 2-1) of the posture improvement assistance system SKS of the second embodiment. FIG. 20B illustrates an operation (part 2-2) of the posture improvement assistance system SKS of the second embodiment. FIG. 20C illustrates an operation (part 2-3) of the posture improvement assistance system SKS of the second embodiment.

FIG. 21A illustrates an operation (part 1) of a posture improvement assistance system SKS of a third embodiment. FIG. 21B illustrates an operation (part 2) of the posture improvement assistance system SKS of the third embodiment. FIG. 21C illustrates an operation (part 3) of the posture improvement assistance system SKS of the third embodiment.

FIG. 22A illustrates an operation (part 1) of a posture improvement assistance system SKS of a fourth embodiment. FIG. 22B illustrates an operation (part 2) of the posture improvement assistance system SKS of the fourth embodiment. FIG. 22C illustrates an operation (part 3) of the posture improvement assistance system SKS of the fourth embodiment. FIG. 22D illustrates an operation (part 4) of the posture improvement assistance system SKS of the fourth embodiment. FIG. 22E illustrates an operation (part 5) of the posture improvement assistance system SKS of the fourth embodiment.

FIG. 23A illustrates an operation (part 1) of a posture improvement assistance system SKS of a fifth embodiment. FIG. 23B illustrates an operation (part 2) of the posture improvement assistance system SKS of the fifth embodiment. FIG. 23C illustrates an operation (part 3) of the posture improvement assistance system SKS of the fifth embodiment.

FIG. 24A illustrates an operation (part 1) of a posture improvement assistance system SKS of a sixth embodiment. FIG. 24B illustrates an operation (part 2) of the posture improvement assistance system SKS of the sixth embodiment.

FIG. 25A illustrates an operation (part 1) of a posture improvement assistance system SKS of a seventh embodiment. FIG. 25B illustrates an operation (part 2) of the posture improvement assistance system SKS of the seventh embodiment. FIG. 25C illustrates an operation (part 3) of the posture improvement assistance system SKS of the seventh embodiment. FIG. 25D illustrates an operation (part 4) of the posture improvement assistance system SKS of the seventh embodiment.

FIG. 26A illustrates an operation (part 1) of a posture improvement assistance system SKS of an eighth embodiment. FIG. 26B illustrates an operation (part 2) of the posture improvement assistance system SKS of the eighth embodiment. FIG. 26C illustrates an operation (part 3) of the posture improvement assistance system SKS of the eighth embodiment. FIG. 26D illustrates an operation (part 4) of the posture improvement assistance system SKS of the eighth embodiment.

FIG. 27A illustrates an operation (part 1) of a posture improvement assistance system SKS of a ninth embodiment. FIG. 27B illustrates an operation (part 2) of the posture improvement assistance system SKS of the ninth embodiment. FIG. 27C illustrates an operation (part 3) of the posture improvement assistance system SKS of the ninth embodiment.

FIG. 28 illustrates an operation of a posture improvement assistance system SKS of Modification 1.

FIG. 29A illustrates an operation (part 1) of a posture improvement assistance system SKS of Modification 2. FIG. 29B illustrates an operation (part 2) of the posture improvement assistance system SKS of Modification 2. FIG. 29C illustrates an operation (part 3) of the posture improvement assistance system SKS of Modification 2. FIG. 29D illustrates an operation (part 4) of the posture improvement assistance system SKS of Modification 2.

### DESCRIPTION OF EMBODIMENTS

Embodiments of the posture improvement assistance system according to the present disclosure will be described.

### First Embodiment

### First Embodiment

A posture improvement assistance system SKS of a first embodiment will be described.

### Function of First Embodiment

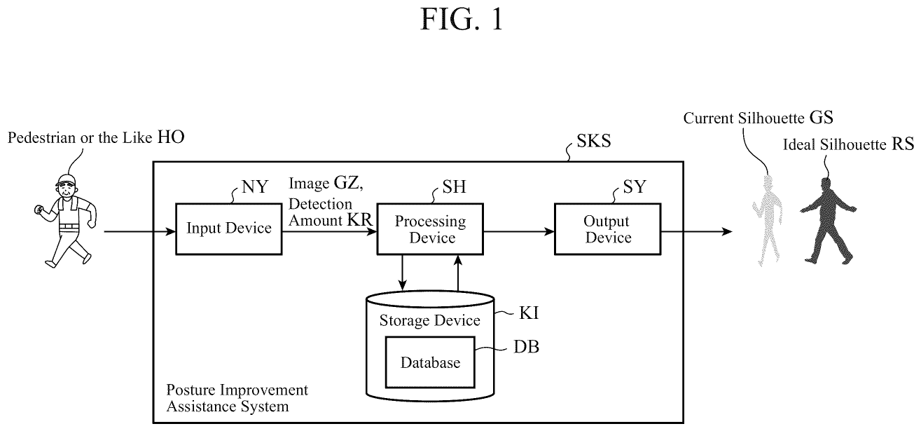

FIG. 1 is a functional block diagram of the posture improvement assistance system SKS of the first embodiment. Hereinafter, a function of the posture improvement assistance system SKS of the first embodiment will be described with reference to FIG. 1.

The posture improvement assistance system SKS of the first embodiment mainly includes an input device NY, a processing device SH, an output device SY, and a storage device KI as illustrated in FIG. 1 in order to assist a pedestrian or the like HO to improve a standing state or a walking motion of the pedestrian or the like HO, for example, by display or the like of a current silhouette GS and an ideal silhouette RS.

Here, the pedestrian or the like HO means, for example, a general term for a walking person and a standing person.

The input device NY corresponds to an “input device”, the processing device SH corresponds to a “processing device”, and the output device SY corresponds to an “output device”.

The pedestrian or the like HO corresponds to a “user”.

The input device NY receives, as user information regarding a posture of the pedestrian or the like HO, input of both an image GZ and a detection amount KR for the pedestrian or the like HO, that is, acquires the image GZ and the detection amount KR of the pedestrian or the like HO.

The processing device SH generates a current silhouette GS representing a current posture of the pedestrian or the like HO on the basis of the user information of the pedestrian or the like HO, that is, the image GZ and the detection amount KR of the pedestrian or the like HO. The processing device SH also extracts an ideal silhouette RS by searching a database DB stored in the storage device KI on the basis of the user information, the current silhouette GS, and the like of the pedestrian or the like HO. Generation of the current silhouette GS, a configuration of the database DB, and the like will be described later.

The output device SY compares one of the current silhouette GS, an improved silhouette KS, and an adapted silhouette TS of the pedestrian or the like HO with the ideal silhouette RS and outputs a result thereof. Details of the current silhouette GS to the ideal silhouette RS will be described later.

The storage device KI stores the database DB.

### <Position of Input Device NY>

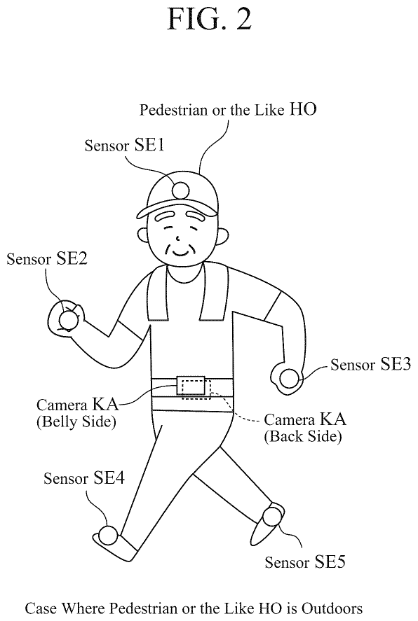

FIG. 2 illustrates a position of the input device NY of the first embodiment (part 1).

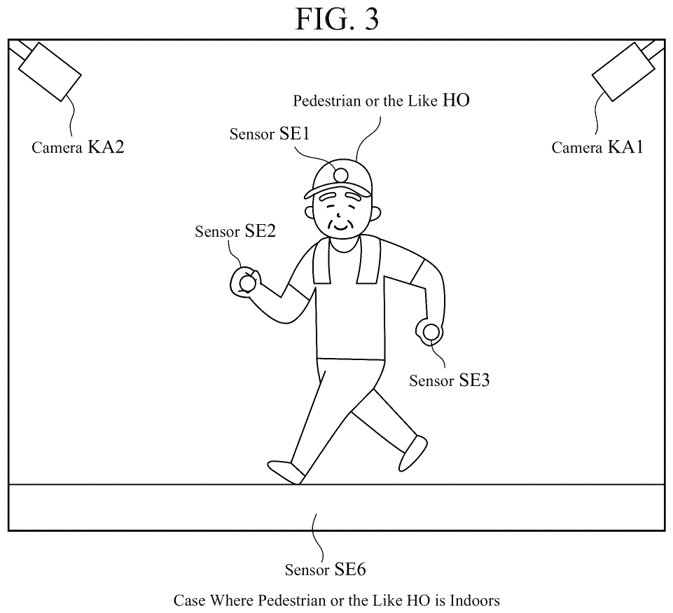

FIG. 3 illustrates a position of the input device NY of the first embodiment (part 2).

In a case where the pedestrian or the like HO is outdoors, as illustrated in FIG. 2, sensors SE1 to SE5 and cameras KA which are the input devices NY are attached to, for example, a cap, wrists, ankles, a belt, and the like of the pedestrian or the like HO. The front body and the rear body of the pedestrian or the like HO are photographed by a camera KA (belly side) and a camera KA (back side) attached to the belt.

In a case where the pedestrian or the like HO is indoors, as illustrated in FIG. 3, the sensors SE1 to SE3 which are the input devices NY are attached to, for example, a cap and wrists of the pedestrian or the like HO, a sensor SE6 which is the input device NY is attached to, for example, the floor in the room, and cameras KA1 and KA2 are attached to, for example, the ceiling in the room (for example, at corners of the ceiling).

Here, each of the sensors SE1 to SE6 is, for example, a position sensor, an acceleration sensor, a human sensor, a vibration sensor, a thermal image sensor, a distance sensor, a pressure sensor, a foot sole load sensor, or a load balance sensor in order to detect a standing state or a walking motion of the pedestrian or the like HO, in other words, in order to acquire the detection amount KR.

The cameras KA, KA1, and KA2 are, for example, wide-angle lens cameras.

### <Position of Output Device SY>

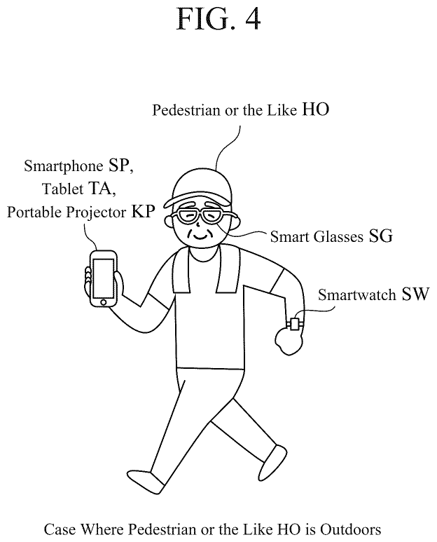

FIG. 4 illustrates a position of the output device SY of the first embodiment (part 1).

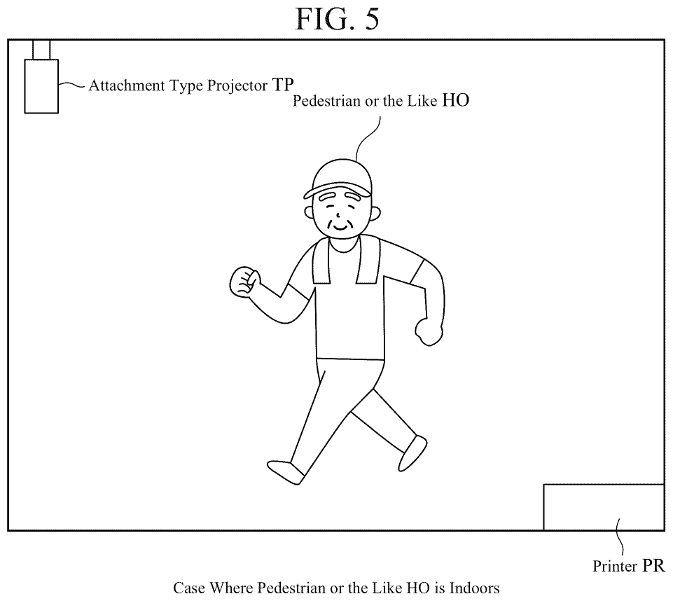

FIG. 5 illustrates a position of the output device SY of the first embodiment (part 2).

In a case where the pedestrian or the like HO is outdoors, as illustrated in FIG. 4, a smartphone SP, a tablet TA, and a portable projector KP which are the output devices SY are, for example, carried by a hand of the pedestrian or the like HO, smart glasses SG which is the output device SY is, for example, worn on a face of the pedestrian or the like HO, and a smartwatch SW which is the output device SY is, for example, attached to a wrist of the pedestrian or the like HO.

In a case where the pedestrian or the like HO is indoors, as illustrated in FIG. 5, an attachment type projector TP which is the output device SY is, for example, attached to the ceiling (for example, a corner of the ceiling) in the room, and a printer PR which is the output device SY is, for example, placed on the floor (for example, a corner of the floor) in the room.

### <Operation of Processing Device SH>

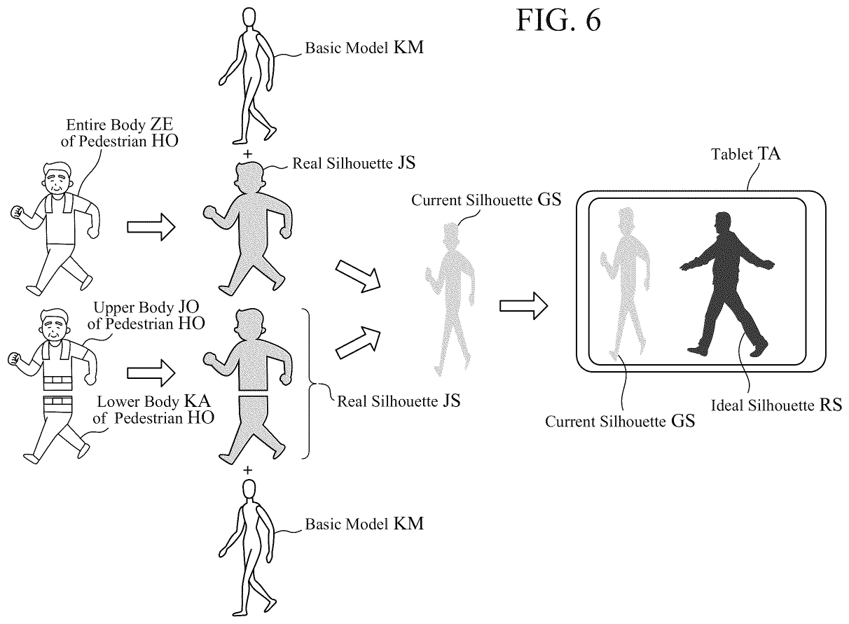

FIG. 6 illustrates an operation of the processing device SH of the first embodiment.

As illustrated in FIG. 6, the processing device SH generates a real silhouette JS indicating a posture of the pedestrian or the like HO from an image GZ of the pedestrian or the like HO captured by the input device NY, more specifically, an image GZ of an entire body ZE of the pedestrian or the like HO or an image GZ of an upper body JO and a lower body KA of the pedestrian or the like HO. After generating the real silhouette JS, the processing device SH generates the current silhouette GS by correcting the real silhouette JS by referring to a basic model KM prepared in advance (for example, stored in advance in the storage device KI). Here, the basic model KM is a standard posture of a human, for example, a posture of medium height and build. The generated current silhouette GS is displayed on, for example, the tablet TA which is the output device SY together with the ideal silhouette RS.

### <Mode of Output Device SY>

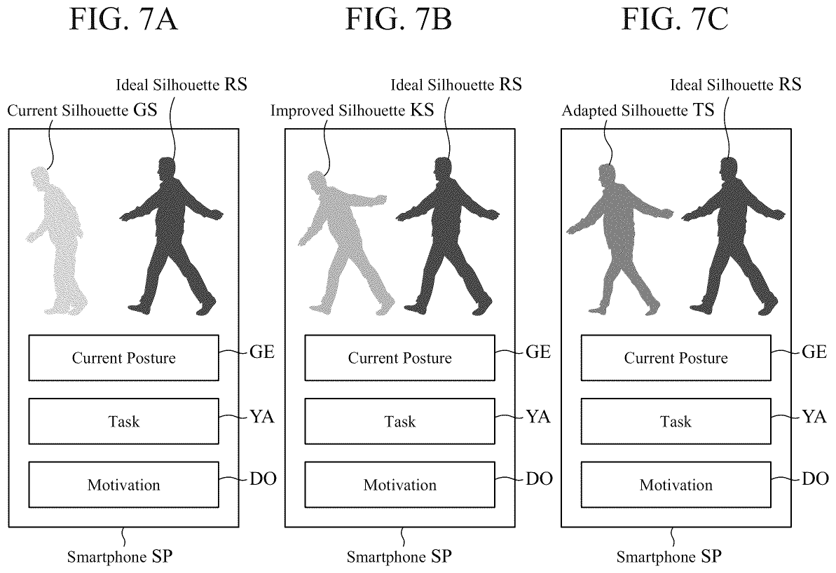

FIG. 7 illustrates a mode output by the output device SY of the first embodiment.

The output device SY basically displays or prints two silhouettes as illustrated in FIGS. 7A to 7C. For example, as a smartphone SP, specifically, the output device SY displays the current silhouette GS and the ideal silhouette RS as illustrated in FIG. 7A, displays the improved silhouette KS and the ideal silhouette RS as illustrated in FIG. 7B, and displays the adapted silhouette TS and the ideal silhouette RS as illustrated in FIG. 7C.

The output device SY may perform printing as the printer PR instead of the display.

Here, the improved silhouette KS refers to an improved posture of the pedestrian or the like HO obtained as a result of improvement of the posture by the pedestrian or the like HO. The adapted silhouette TS refers to a posture of the pedestrian or the like HO obtained as a result of further improvement of the posture by the pedestrian or the like HO and adapted to a target posture, that is, the ideal silhouette RS.

Hereinafter, in order to facilitate description and understanding, “the current silhouette GS, the ideal silhouette RS, and the like” refers to a combination of the current silhouette GS and the ideal silhouette RS, a combination of the improved silhouette KS and the ideal silhouette RS, and a combination of the adapted silhouette TS and the ideal silhouette RS.

For example, as a smartphone SP, as illustrated in FIGS. 7A to 7C, the output device SY additionally displays a current posture GE, a task YA, and a motivation DO under the two silhouettes, that is, the current silhouette GS, the ideal silhouette RS, and the like. Details of the current posture GE, the task YA, and the motivation DO will be described later.

### <Type of Output Device SY>

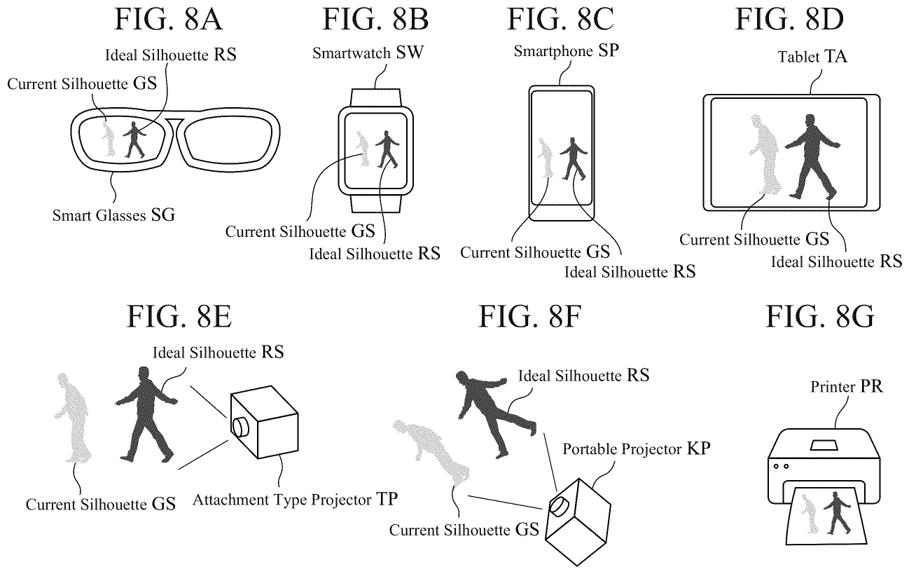

FIG. 8 illustrates a type of the output device SY of the first embodiment.

For example, as illustrated in FIGS. 8A to 8F, the output device SY is smart glasses SG, a smartwatch SW, a smartphone SP, a tablet TA, an attachment type projector TP, or a portable projector KP that displays the current silhouette GS, the ideal silhouette RS, and the like (also illustrated in FIG. 7), and as illustrated in FIG. 8G, the output device SY is a printer PR that prints the current silhouette GS, the ideal silhouette RS, and the like.

In particular, the portable projector KP is carried by the pedestrian or the like HO in the outdoors (illustrated in FIG. 4), and projects the current silhouette GS, the ideal silhouette RS, and the like, for example, toward a road in the outdoors. The attachment type projector TP is attached in a room (illustrated in FIG. 5), and projects the current silhouette GS, the ideal silhouette RS, and the like, for example, toward a wall in the room.

### <Projection of Output Device SY>

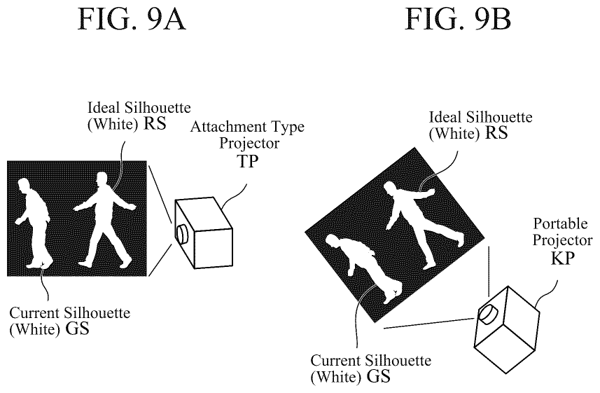

FIG. 9 illustrates one mode of projection of the output device SY of the first embodiment.

Instead of a normal silhouette, that is, the current silhouette GS, the ideal silhouette RS, and the like (for example, illustrated in FIG. 7A to FIG. 7C), the attachment type projector TP which is the output device SY may project a current silhouette (white) GS which is white, an ideal silhouette RS (white) which is white, and the like, for example, as illustrated in FIG. 9A. Similarly, instead of a normal silhouette, that is, the current silhouette GS, the ideal silhouette RS, and the like (for example, illustrated in FIG. 7A to FIG. 7C), the attachment type projector TP which is the output device SY may project a current silhouette GS (white) which is white, an ideal silhouette RS (white) which is white, and the like as illustrated in FIG. 9B.

### <Alternatives to Current Silhouette GS and the Like>

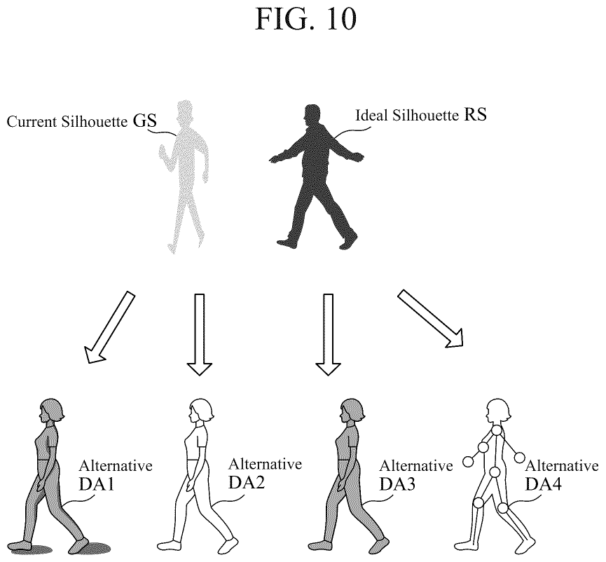

FIG. 10 illustrates alternatives to the current silhouette GS, the ideal silhouette RS, and the like of the first embodiment.

Each of the current silhouette GS, the ideal silhouette RS, and the like may be, for example, an alternative DA1 (real moving image), an alternative DA2 (3D moving image), an alternative DA3 (2D animation), or an alternative DA4 (bone pattern (registered trademark) indicating a skeleton). In FIG. 10, in order to indicate that the gender is irrelevant, the alternatives DA1 to DA4 represent women in contrast to the current silhouette GS and the ideal silhouette RS representing men.

### <Configuration of Database DB>

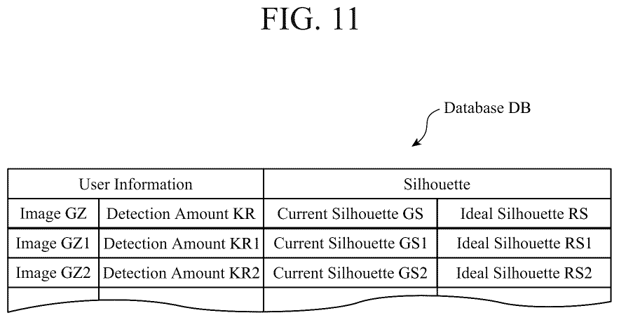

FIG. 11 illustrates a configuration of a database DB of the first embodiment.

In the database DB of the first embodiment, user information and a silhouette are associated with each other as illustrated in FIG. 11. In the database DB, more specifically, an image GZ of the pedestrian or the like HO, a detection amount KR of the pedestrian or the like HO, a current silhouette GS of the pedestrian or the like HO, and an ideal silhouette RS corresponding to the current silhouette GS are associated with each other.

For example, when an image GZ1 of the pedestrian or the like HO is captured and a detection amount KR1 of the pedestrian or the like HO is acquired by the input device NY, and a current silhouette GS1 is generated by the processing device SH, the processing device SH extracts an ideal silhouette RS1 corresponding to the current silhouette GS1 by searching the database DB on the basis of at least one or more of the image GZ1, the detection amount KR1, and the current silhouette GS1.

Similarly, for example, when an image GZ2 of the pedestrian or the like HO is captured and a detection amount KR2 of the pedestrian or the like HO is acquired by the input device NY, and a current silhouette GS2 is generated by the processing device SH, the processing device SH extracts an ideal silhouette RS2 corresponding to the current silhouette GS2 by searching the database DB on the basis of at least one or more of the image GZ2, the detection amount KR2, and the current silhouette GS2.

### <Configuration of Processing Device SH>

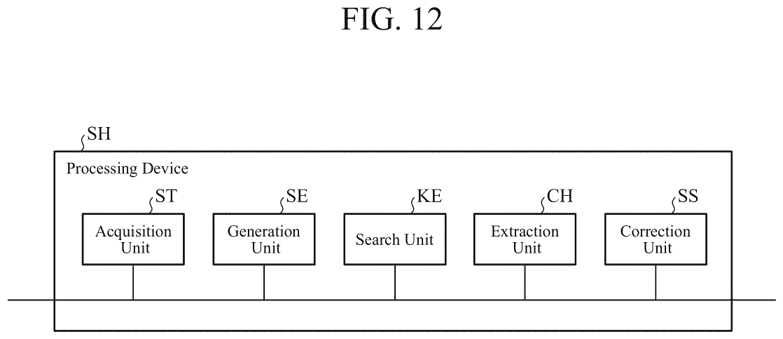

FIG. 12 is a functional block diagram of the processing device SH of the first embodiment.

As illustrated in FIG. 12, the processing device SH of the first embodiment includes an acquisition unit ST, a generation unit SE, a search unit KE, an extraction unit CH, and a correction unit SS.

The acquisition unit ST corresponds to an “acquisition unit”, the generation unit SE corresponds to a “generation unit”, the search unit KE corresponds to a “search unit”, the extraction unit CH corresponds to an “extraction unit”, and the correction unit SS corresponds to a “correction unit”.

The acquisition unit ST acquires user information that is information regarding a posture of the pedestrian or the like HO, that is, an image GZ and a detection amount KR of the pedestrian or the like HO.

The generation unit SE generates a current silhouette GS representing a current posture of the pedestrian or the like HO on the basis of the user information of the pedestrian or the like HO. The generation unit SE also generates a real silhouette JS representing the pedestrian or the like HO from the image GZ of the pedestrian or the like HO.

The generation unit SE generates the current silhouette GS, for example, by the correction unit SS correcting the real silhouette JS by referring to a basic model KM.

The search unit KE searches the database DB (illustrated in FIG. 11) on the basis of at least one or more pieces of the user information of the pedestrian or the like HO, that is, the image GZ of the pedestrian or the like HO, the detection amount KR of the pedestrian or the like HO, and the current silhouette GS of the pedestrian or the like HO.

The extraction unit CH extracts an ideal silhouette RS corresponding to the current silhouette GS of the pedestrian or the like HO, found by the search of the search unit KE.

The correction unit SS corrects the real silhouette JS by referring to the basic model KM (FIG. 6).

### Hardware Configuration and Software-Based Configuration of First Embodiment

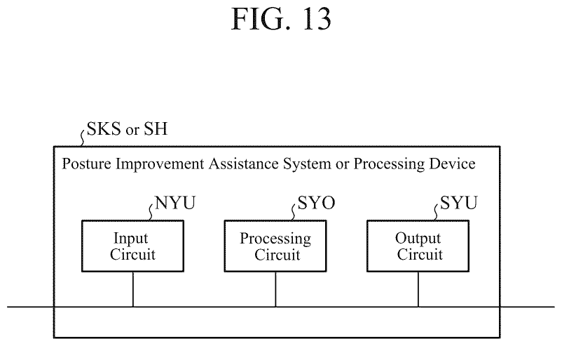

FIG. 13 illustrates a hardware configuration of each of the posture improvement assistance system SKS and the processing device SH of the first embodiment.

As illustrated in FIG. 13, each of the posture improvement assistance system SKS and the processing device SH of the first embodiment includes a processing circuit SYO, and further include an input circuit NYU and an output circuit SYU as necessary in order to perform the functions.

The processing circuit SYO is dedicated hardware. The processing circuit SYO implements functions of the acquisition unit ST to the correction unit SS (illustrated in FIG. 12) of the processing device SH of the posture improvement assistance system SKS.

The processing circuit SYO is, for example, a single circuit, a composite circuit, a programmed processor, a parallel programmed processor, an application specific integrated circuit (ASIC), a field programmable gate array (FPGA), or a combination thereof.

The input circuit NYU and the output circuit SYU exchange input and output related to an operation of the processing circuit SYO with, for example, the outside of the posture improvement assistance system SKS or the outside of the processing device SH.

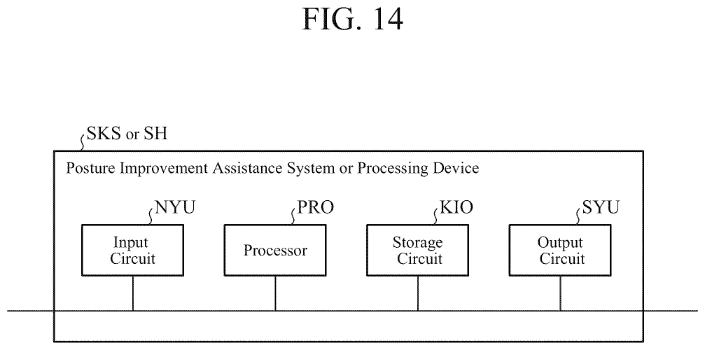

FIG. 14 illustrates a hardware configuration based on software implementation of the posture improvement assistance system SKS and the processing device SH of the embodiment.

As illustrated in FIG. 14, each of the posture improvement assistance system SKS and the processing device SH of the first embodiment includes a processor PRO and a storage circuit KIO, and further includes the input circuit NYU and the output circuit SYU as necessary.

The processor PRO is a central processing unit (CPU, also referred to as a central processing device, a processing device, an arithmetic device, a microprocessor, a microcomputer, or a digital signal processor (DSP)) that executes a program. The processor PRO implements functions of the acquisition unit ST to the correction unit SS (illustrated in FIG. 12) of the processing device SH of the posture improvement assistance system SKS.

The processor PRO implements the functions by software, firmware, or a combination of software and firmware. Each of software and firmware is described as a program and stored in the storage circuit KIO.

The processor PRO implements the functions by reading and executing the program from the storage circuit KIO. It can be said that the program causes a computer to execute procedures and methods performed by the acquisition unit ST to the correction unit SS of the processing device SH of the posture improvement assistance system SKS.

Here, the storage circuit KIO is, for example, a nonvolatile or volatile semiconductor memory such as random access memory (RAM), read only memory (ROM), flash memory, erasable programmable read only memory (EPROM), or electrically erasable programmable read-only memory (EEPROM), a magnetic disk, a flexible disk, an optical disc, a compact disc, a mini disc, or a digital versatile disc (DVD).

Some of the functions of the acquisition unit ST to the correction unit SS of the processing device SH of the posture improvement assistance system SKS may be implemented by the processing circuit SYO (illustrated in FIG. 13), while some other functions may be implemented by the processor PRO (illustrated in FIG. 14).

As described above, the functions of the acquisition unit ST to the correction unit SS of the processing device SH of the posture improvement assistance system SKS can be implemented by hardware, software, firmware, or a combination thereof.

The input unit NYU and the output unit SYU exchange input and output related to an operation of the processor PRO with, for example, the outside of the posture improvement assistance system SKS or the outside of the processing device SH.

### Operation of First Embodiment

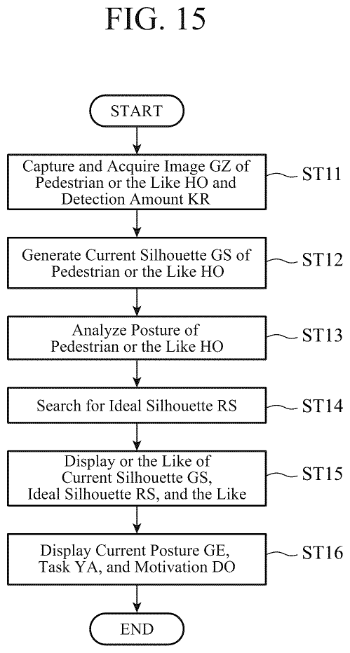

FIG. 15 is a flowchart illustrating an operation of the posture improvement assistance system SKS of the first embodiment.

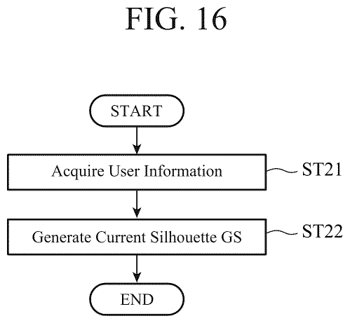

FIG. 16 is a flowchart illustrating an operation of the processing device SH of the first embodiment.

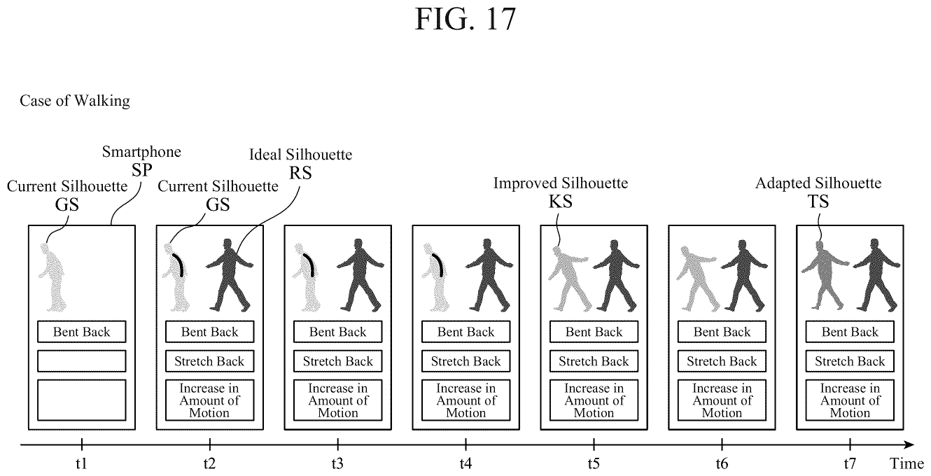

FIG. 17 illustrates a mode (part 1) of display or the like of the posture improvement assistance system SKS of the first embodiment.

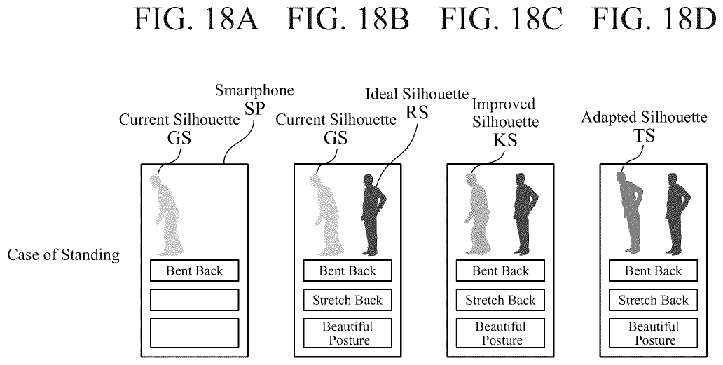

FIG. 18 illustrates a mode (part 2) of the display or the like of the posture improvement assistance system SKS of the first embodiment.

An operation of the posture improvement assistance system SKS and an operation of the processing device SH of the first embodiment will be described with reference to FIGS. 15 to 18.

Step ST11: The input device NY (illustrated in FIG. 1) captures an image GZ of the pedestrian or the like HO (illustrated in FIG. 1), and acquires a standing state or a walking motion of the pedestrian or the like HO, that is, a detection amount KR. In other words, in the processing device SH, in step ST21 which is an operation of the processing device SH, the acquisition unit ST acquires the image GZ and the detection amount KR of the pedestrian or the like HO which are user information of the pedestrian or the like HO via the input device NY.

Step ST12: As illustrated in FIG. 6, the processing device SH (illustrated in FIG. 1) generates a real silhouette JS of the pedestrian or the like HO from the image GZ of the pedestrian or the like HO, and further generates a current silhouette GS of the pedestrian or the like HO by using the real silhouette JS of the pedestrian or the like HO and a basic model KM. In summary, in the processing device SH, the generation unit SE generates the current silhouette GS by the procedure in step ST22 which is an operation of the processing device SH.

Step ST13: The processing device SH analyzes a posture of the pedestrian or the like HO, for example, a standing state or a walking motion on the basis of the captured image GZ of the pedestrian or the like HO and the acquired detection amount KR of the pedestrian or the like HO.

Step ST14: The processing device SH extracts an ideal silhouette RS corresponding to the current silhouette GS of the pedestrian or the like HO by searching the database DB (illustrated in FIG. 11) in the storage device KI on the basis of user information of the pedestrian or the like HO, that is, the image GZ and the detection amount KR of the pedestrian or the like HO.

Step ST15: For example, when the pedestrian or the like HO is walking, the output device SY (illustrated in FIG. 1) which is a smartphone SP displays the current silhouette GS, the ideal silhouette RS, and the like of the pedestrian or the like HO in time series, that is, continuously, and displays the current silhouette GS, the ideal silhouette RS, and the like of the pedestrian or the like HO separately from each other as illustrated in FIG. 17.

More specifically, the output device SY displays only the current silhouette GS of the pedestrian or the like HO at time t1, that is, at an initial stage. The output device SY displays the current silhouette GS and the ideal silhouette RS of the pedestrian or the like HO at times t2 to t4. The output device SY displays an improved silhouette KS and the ideal silhouette RS of the pedestrian or the like HO at times t5 to t6. The output device SY displays an adapted silhouette TS and the ideal silhouette RS of the pedestrian or the like HO at time t7.

On the other hand, when the pedestrian or the like HO is standing, as illustrated in FIG. 18, the output device SY displays the current silhouette GS, the ideal silhouette RS, and the like of the pedestrian or the like HO in a snapshot manner, that is, in a single manner, and displays the current silhouette GS, the ideal silhouette RS, and the like of the pedestrian or the like HO separately from each other.

More specifically, the output device SY displays only the current silhouette GS of the pedestrian or the like HO at an initial stage as illustrated in FIG. 18A. Thereafter, as illustrated in FIG. 18B, the output device SY displays the current silhouette GS and the ideal silhouette RS of the pedestrian or the like HO. Thereafter, as illustrated in FIG. 18C, the output device SY further displays the improved silhouette KS and the ideal silhouette RS of the pedestrian or the like HO. Finally, as illustrated in FIG. 18D, the output device SY displays the adapted silhouette TS and the ideal silhouette RS of the pedestrian or the like HO.

Step ST16: The output device SY displays a current posture GE, a task YA, and a motivation DO in addition to display or the like of the current silhouette GS, the ideal silhouette RS, and the like.

Here, the current posture GE is a current posture of the pedestrian or the like HO, that is, a current posture. The task YA is an action that the pedestrian or the like HO should perform for improvement of the current posture by the pedestrian or the like HO. The motivation DO is a motivation for the pedestrian or the like HO to execute the task YA.

When the pedestrian or the like HO is walking, as illustrated in FIG. 17, the output device SY additionally displays the current posture GE “bent back” at time t1, that is, at an initial stage. From time t2 to time t7, the output device SY additionally displays the current posture GE “bent back”, the task YA “stretch back”, and the motivation DO “increase in amount of motion”.

When the pedestrian or the like HO is standing, the output device SY additionally displays the current posture GE “bent back” at an initial stage as illustrated in FIG. 18A. From then to the end, as illustrated in FIGS. 18B to 18D, the output device SY additionally displays the current posture GE “bent back”, the task YA “stretch back”, and the motivation DO “beautiful posture”.

### Effects of First Embodiment

As described above, in the posture improvement assistance system SKS of the first embodiment, the output device SY displays a current silhouette GS, an ideal silhouette RS, and the like, that is, displays or prints a current silhouette GS indicating a current posture of the pedestrian or the like HO and an ideal silhouette RS to be aimed by the pedestrian or the like HO, an improved silhouette KS indicating an improved posture of the pedestrian or the like HO and the ideal silhouette RS, or an adapted silhouette TS indicating an adapted posture of the pedestrian or the like HO and the ideal silhouette RS. As a result, the pedestrian or the like HO compares a posture indicated by the ideal silhouette RS with a current posture, an improved posture, or an adapted posture of the pedestrian or the like HO, indicated by the current silhouette GS, the improved silhouette KS, or the adapted silhouette TS. As a result, the pedestrian or the like HO can easily understand how the posture of the pedestrian or the like HO is bad, how the posture of the pedestrian or the like HO is improved, and how the posture of the pedestrian or the like HO is adapted as compared with related art described with a technical content, and as a result, a standing position and walking of the pedestrian or the like HO can be easily improved as compared with related art.

In the posture improvement assistance system SKS of the first embodiment, the output device SY additionally performs display or the like of the current posture GE, the task YA, and the motivation DO in addition to display or the like of the current silhouette GS, the ideal silhouette RS, and the like of the pedestrian or the like HO. As a result, the pedestrian or the like HO can more accurately grasp the current posture represented by the current silhouette GS of the pedestrian or the like HO, can recognize a task in order to improve the posture of the pedestrian or the like HO, and can enhance a motivation for practicing the task YA.

### Second Embodiment

### Second Embodiment

A posture improvement assistance system SKS of a second embodiment will be described.

The posture improvement assistance system SKS of the second embodiment is different from the posture improvement assistance system SKS of the first embodiment that assists improvement of a standing state and a walking motion of a pedestrian or the like HO, and assists improvement of a stride of the pedestrian or the like HO.

### Function, Hardware Configuration, and Software-Based Configuration of Second Embodiment

A function, a hardware configuration, and a software-based configuration of the posture improvement assistance system SKS of the second embodiment are similar to the function, the hardware configuration, and the software-based configuration of the posture improvement assistance system SKS of the first embodiment (illustrated in FIGS. 1, 13, and 14).

### Operation of Second Embodiment

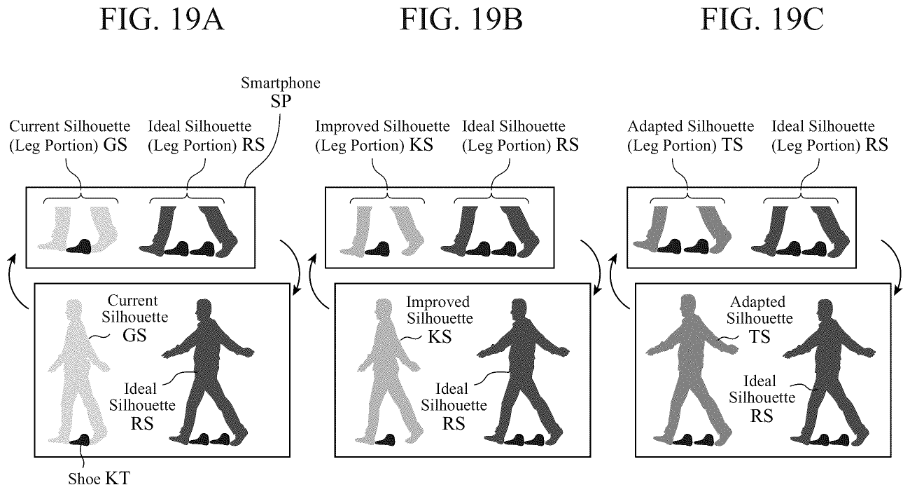

FIG. 19 illustrates an operation (part 1) of the posture improvement assistance system SKS of the second embodiment.

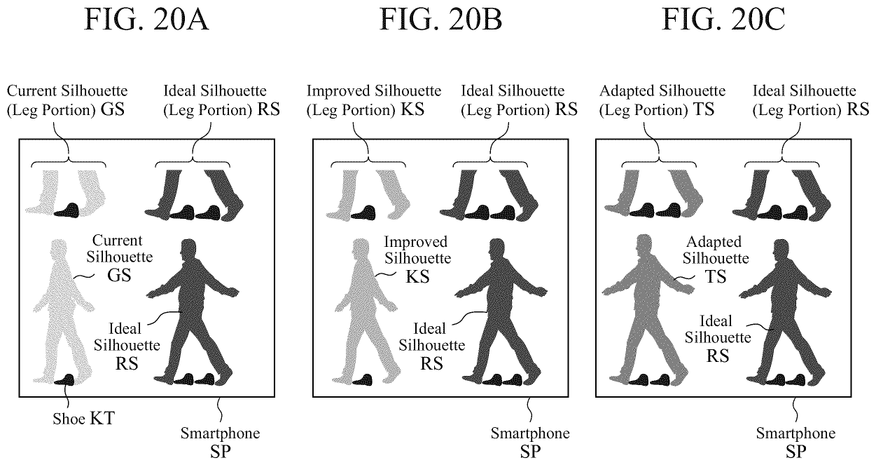

FIG. 20 illustrates an operation (part 2) of the posture improvement assistance system SKS of the second embodiment.

The operation of the posture improvement assistance system SKS of the second embodiment will be described with reference to FIGS. 19 and 20.

The operation of the posture improvement assistance system SKS of the second embodiment is basically similar to the operation of the posture improvement assistance system SKS of the first embodiment.

On the other hand, unlike the posture improvement assistance system SKS of the first embodiment, in the posture improvement assistance system SKS of the second embodiment, an output device SY (illustrated in FIG. 1) performs display or the like of a shoe KT in addition to display or the like of a current silhouette GS, an ideal silhouette RS, and the like.

Here, the shoe KT is one of symbols representing a current posture of the pedestrian or the like HO.

In the posture improvement assistance system SKS of the second embodiment, more specifically, as illustrated in FIGS. 19A to 19C and 20A to 20C, the output device SY indicates that an ideal stride is the length of two shoes KT from a relationship between the ideal silhouette RS and the two shoes KT.

In the posture improvement assistance system SKS of the second embodiment, on the other hand, (1) it is indicated that a stride of the current silhouette GS is extremely narrower than the length of two shoes KT from a relationship between the current silhouette GS and one shoe KT as illustrated in FIGS. 19A and 20A, (2) it is indicated that a stride of an improved silhouette KS is narrower than the length of two shoes KT from a relationship between the improved silhouette KS and one shoe KT as illustrated in FIGS. 19B and 20B, and (3) it is indicated that a stride of an adapted silhouette TS is slightly narrower than the length of two shoes KT from a relationship between the adapted silhouette TS and the two shoes KT as illustrated in FIGS. 19C and 20C.

In the posture improvement assistance system SKS of the second embodiment, the output device SY switches between display or the like of a partial silhouette and display or the like of an entire silhouette, and on the other hand, collectively performs display or the like of the partial silhouette and display or the like of the entire silhouette.

More specifically, regarding the former display or the like by switching, unlike the posture improvement assistance system SKS of the first embodiment, in the posture improvement assistance system SKS of the second embodiment, in step ST15 (illustrated in FIG. 15), as illustrated in FIG. 19A, the output device SY which is a smartphone SP switches between displaying a current silhouette (leg portion) GS indicating a current stride of the pedestrian or the like HO and an ideal silhouette (leg portion) RS indicating an ideal stride of the pedestrian or the like HO and displaying a current silhouette GS indicating a current posture of the pedestrian or the like HO himself/herself and an ideal silhouette RS indicating a posture which should be ideal for the pedestrian or the like HO.

Similarly, in the posture improvement assistance system SKS of the second embodiment, in the step ST15, as illustrated in FIG. 19B, the output device SY which is a smartphone SP switches between displaying an improved silhouette (leg portion) KS indicating an improved stride of the pedestrian or the like HO and an ideal silhouette (leg portion) RS indicating an ideal stride of the pedestrian or the like HO and displaying an improved silhouette KS indicating an improved posture of the pedestrian or the like HO himself/herself and an ideal silhouette RS indicating a posture which should be ideal for the pedestrian or the like HO.

Similarly, in the posture improvement assistance system SKS of the second embodiment, in the step ST15, as illustrated in FIG. 19C, the output device SY which is a smartphone SP switches between displaying an ideal silhouette (leg portion) TS indicating an adapted stride of the pedestrian or the like HO and an ideal silhouette (leg portion) RS indicating an ideal stride of the pedestrian or the like HO and displaying an adapted silhouette TS indicating an adapted posture of the pedestrian or the like HO himself/herself and an ideal silhouette RS indicating a posture which should be ideal for the pedestrian or the like HO.

More specifically, regarding the latter collective display or the like, in the posture improvement assistance system SKS of the second embodiment, in step ST15, instead of display or the like illustrated in FIG. 19, as illustrated in FIG. 20A, the output device SY which is a smartphone SP performs collective display or the like of a current silhouette (leg portion) GS indicating a current stride of the pedestrian or the like HO, an ideal silhouette (leg portion) RS indicating an ideal stride of the pedestrian or the like HO, a current silhouette GS indicating a current posture of the pedestrian or the like HO himself/herself, and an ideal silhouette RS indicating a posture which should be ideal for the pedestrian or the like HO.

Similarly, in the posture improvement assistance system SKS of the second embodiment, in step ST15, as illustrated in FIG. 20B, the output device SY which is a smartphone SP performs collective display or the like of an improved silhouette (leg portion) KS indicating an improved stride of the pedestrian or the like HO, an ideal silhouette (leg portion) RS indicating an ideal stride of the pedestrian or the like HO, an improved silhouette KS indicating an improved posture of the pedestrian or the like HO himself/herself, and an ideal silhouette RS indicating a posture which should be ideal for the pedestrian or the like HO.

Similarly, in the posture improvement assistance system SKS of the second embodiment, in step ST15, as illustrated in FIG. 20C, the output device SY which is a smartphone SP performs collective display or the like of an adapted silhouette (leg portion) TS indicating an adapted stride of the pedestrian or the like HO, an ideal silhouette (leg portion) RS indicating an ideal stride of the pedestrian or the like HO, an adapted silhouette TS indicating an adapted posture of the pedestrian or the like HO himself/herself, and an ideal silhouette RS indicating a posture which should be ideal for the pedestrian or the like HO.

### Effects of Second Embodiment

As described above, in the posture improvement assistance system SKS of the second embodiment, the output device SY simultaneously performs display or the like of the current silhouette GS, the ideal silhouette RS, and the like, and the shoe KT. As a result, the pedestrian or the like HO can easily understand how different a stride of the pedestrian or the like HO is from an ideal stride, how different an improved stride of the pedestrian or the like HO is from the ideal stride, and how different an adapted stride of the pedestrian or the like HO is from the ideal stride as compared with a related art.

### Third Embodiment

### Third Embodiment

A posture improvement assistance system SKS of a third embodiment will be described.

The posture improvement assistance system SKS of the third embodiment is different from the posture improvement assistance system SKS of the first embodiment that assists improvement of a standing state and a walking motion of a pedestrian or the like HO, and assists improvement of an arm swing of the pedestrian or the like HO.

### Function, Hardware Configuration, and Software-Based Configuration of Third Embodiment

A function, a hardware configuration, and a software-based configuration of the posture improvement assistance system SKS of the third embodiment are similar to the function, the hardware configuration, and the software-based configuration of the posture improvement assistance system SKS of the first embodiment (illustrated in FIGS. 1, 13, and 14).

### Operation of Third Embodiment

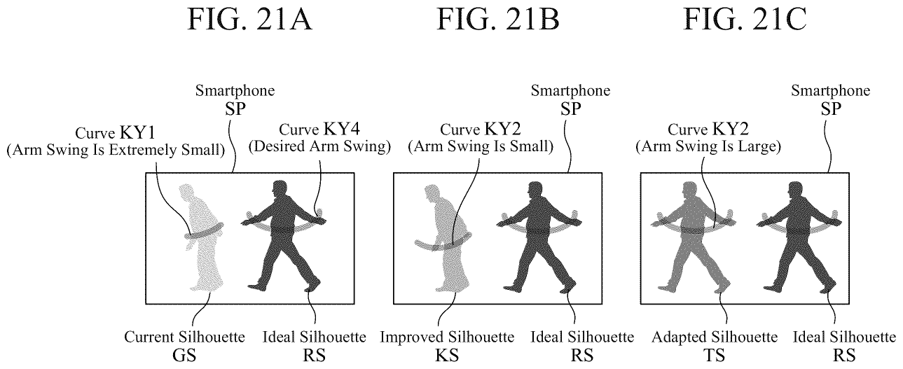

FIG. 21 illustrates an operation of the posture improvement assistance system SKS of the third embodiment.

The operation of the posture improvement assistance system SKS of the third embodiment will be described with reference to FIG. 21.

The operation of the posture improvement assistance system SKS of the third embodiment is basically similar to the operation of the posture improvement assistance system SKS of the first embodiment.

On the other hand, unlike the posture improvement assistance system SKS of the first embodiment, in the posture improvement assistance system SKS of the third embodiment, an output device SY which is a smartphone SP (illustrated in FIG. 1) performs display or the like of a curve KY in addition to display or the like of a current silhouette GS, an ideal silhouette RS, and the like.

Here, the curve KY is one of symbols representing a current posture of the pedestrian or the like HO.

In the posture improvement assistance system SKS of the third embodiment, in step ST15 (illustrated in FIG. 15), as illustrated in FIG. 21A, the output device SY teaches the pedestrian or the like HO that an arm swing of the pedestrian or the like HO is extremely small by performing display or the like of a current silhouette GS having a curve KY1 indicating that the arm swing of the pedestrian or the like HO is extremely small in comparison with an ideal silhouette RS having a curve KY4 indicating a desired arm swing.

In the posture improvement assistance system SKS of the third embodiment, in the step ST15, as illustrated in FIG. 21B, the output device SY which is a smartphone SP teaches the pedestrian or the like HO that an arm swing of the pedestrian or the like HO is small by performing display or the like of an improved silhouette KS having a curve KY2 indicating that the arm swing of the pedestrian or the like HO is small in comparison with the ideal silhouette RS having the curve KY4 indicating a desired arm swing.

In the posture improvement assistance system SKS of the third embodiment, in the step ST15, as illustrated in FIG. 21C, the output device SY which is a smartphone SP teaches the pedestrian or the like HO that an arm swing of the pedestrian or the like HO is large by performing display or the like of an adapted silhouette TS having a curve KY3 indicating that the arm swing of the pedestrian or the like HO is large in comparison with the ideal silhouette RS having the curve KY4 indicating a desired arm swing.

### Effects of Third Embodiment

As described above, in the posture improvement assistance system SKS of the third embodiment, the output device SY performs display or the like of the current silhouette GS, the improved silhouette KS, and the adapted silhouette TS respectively having the curves KY1 to KY3 indicating a state of an arm swing of the pedestrian or the like HO in comparison with the ideal silhouette RS having the curve KY4 indicating a desired arm swing. As a result, the pedestrian or the like HO can easily understand how different the arm swing of the pedestrian or the like HO is from the ideal arm swing.

### Fourth Embodiment

### Fourth Embodiment

A posture improvement assistance system SKS of a fourth embodiment will be described.

The posture improvement assistance system SKS of the fourth embodiment is different from the posture improvement assistance system SKS of the first embodiment that assists improvement of a standing state and a walking motion of a pedestrian or the like HO, and assists improvement of a way of standing of the pedestrian or the like HO.

### Function, Hardware Configuration, and Software-Based Configuration of Fourth Embodiment

A function, a hardware configuration, and a software-based configuration of the posture improvement assistance system SKS of the fourth embodiment are similar to the function, the hardware configuration, and the software-based configuration of the posture improvement assistance system SKS of the first embodiment (illustrated in FIGS. 1, 13, and 14).

### Operation of Fourth Embodiment

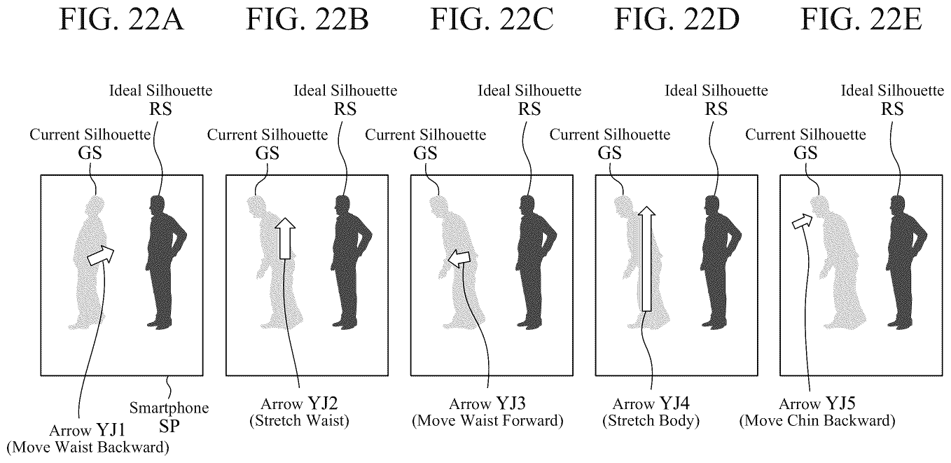

FIG. 22 illustrates an operation of the posture improvement assistance system SKS of the fourth embodiment.

The operation of the posture improvement assistance system SKS of the fourth embodiment will be described with reference to FIG. 22.

The operation of the posture improvement assistance system SKS of the fourth embodiment is basically similar to the operation of the posture improvement assistance system SKS of the first embodiment.

On the other hand, unlike the posture improvement assistance system SKS of the first embodiment, in the posture improvement assistance system SKS of the fourth embodiment, an output device SY which is a smartphone SP (illustrated in FIG. 1) performs display or the like of an arrow YJ in addition to display or the like of a current silhouette GS, an ideal silhouette RS, and the like.

Here, the arrow YJ is one of symbols representing an action for improving a current posture of the pedestrian or the like HO.

In the posture improvement assistance system SKS of the fourth embodiment, in step ST15 (illustrated in FIG. 15), as illustrated in FIG. 22A, the output device SY teaches the pedestrian or the like HO that the waist of the pedestrian or the like HO should be moved backward by performing display or the like of a current silhouette GS having an arrow YJ1 indicating that the waist of the pedestrian or the like HO should be moved backward in comparison with an ideal silhouette RS which is a desired way of standing.

In the posture improvement assistance system SKS of the fourth embodiment, in the step ST15, as illustrated in FIG. 22B, the output device SY teaches the pedestrian or the like HO that the waist of the pedestrian or the like HO should be stretched by performing display or the like of a current silhouette GS having an arrow YJ2 indicating that the waist of the pedestrian or the like HO should be stretched in comparison with the ideal silhouette RS which is a desired way of standing.

In the posture improvement assistance system SKS of the fourth embodiment, in the step ST15, as illustrated in FIG. 22C, the output device SY teaches the pedestrian or the like HO that the waist of the pedestrian or the like HO should be moved forward by performing display or the like of a current silhouette GS having an arrow YJ3 indicating that the waist of the pedestrian or the like HO should be moved forward in comparison with the ideal silhouette RS which is a desired way of standing.

In the posture improvement assistance system SKS of the fourth embodiment, in the step ST15, as illustrated in FIG. 22D, the output device SY teaches the pedestrian or the like HO that a body of the pedestrian or the like HO should be stretched by performing display or the like of a current silhouette GS having an arrow YJ4 indicating that the body of the pedestrian or the like HO should be stretched in comparison with the ideal silhouette RS which is a desired way of standing.

In the posture improvement assistance system SKS of the fourth embodiment, in the step ST15, as illustrated in FIG. 22E, the output device SY teaches the pedestrian or the like HO that the chin of the pedestrian or the like HO should be moved backward by performing display or the like of a current silhouette GS having an arrow YJ5 indicating that the chin of the pedestrian or the like HO should be moved backward in comparison with the ideal silhouette RS which is a desired way of standing.

### Effects of Fourth Embodiment

As described above, the posture improvement assistance system SKS of the fourth embodiment displays the current silhouettes GS having the respective arrows YJ1 to YJ5 indicating actions to be performed by the pedestrian or the like HO in comparison with the ideal silhouette RS which is a desired way of standing. As a result, the pedestrian or the like HO can easily understand how to improve the posture of the pedestrian or the like HO.

### Fifth Embodiment

### Fifth Embodiment

A posture improvement assistance system SKS of a fifth embodiment will be described.

The posture improvement assistance system SKS of the fifth embodiment is different from the posture improvement assistance system SKS of the first embodiment that assists improvement of a standing state and a walking motion of a pedestrian or the like HO, and assists improvement of weight movement of the pedestrian or the like HO.

### Function, hardware configuration, and software-based configuration of Fifth Embodiment

A function, a hardware configuration, and a software-based configuration of the posture improvement assistance system SKS of the fifth embodiment are similar to the function, the hardware configuration, and the software-based configuration of the posture improvement assistance system SKS of the first embodiment (illustrated in FIGS. 1, 13, and 14).

### Operation of Fifth Embodiment

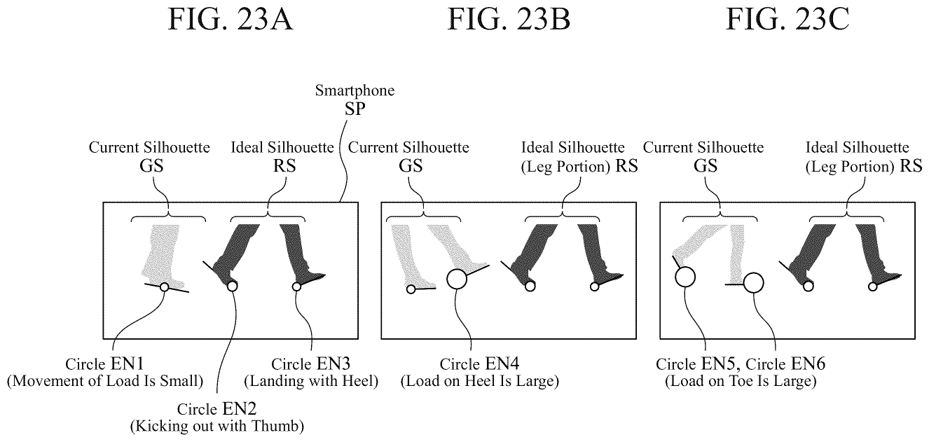

FIG. 23 illustrates an operation of the posture improvement assistance system SKS of the fifth embodiment.

The operation of the posture improvement assistance system SKS of the fifth embodiment will be described with reference to FIG. 23.

The operation of the posture improvement assistance system SKS of the fifth embodiment is basically similar to the operation of the posture improvement assistance system SKS of the first embodiment.

On the other hand, unlike the posture improvement assistance system SKS of the first embodiment, in the posture improvement assistance system SKS of the fifth embodiment, an output device SY (illustrated in FIG. 1) performs display or the like of a circle EN in addition to display or the like of a current silhouette GS, an ideal silhouette RS, and the like.

Here, the circle EN is one of symbols representing a current posture of the pedestrian or the like HO.

In the posture improvement assistance system SKS of the fifth embodiment, as illustrated in FIGS. 23A to 23C, the positions of the circles EN1 to EN6 indicate the positions of load, the sizes of the circles EN1 to EN6 indicate the magnitudes of the load, and a straight line indicates movement of each of the load.

In the posture improvement assistance system SKS of the fifth embodiment, as illustrated in FIG. 23A, the output device SY which is a smartphone SP teaches the pedestrian or the like HO that movement of the load of the pedestrian or the like HO is small by performing display or the like of a current silhouette GS having a circle EN1 indicating that the movement of the load is small in comparison with an ideal silhouette RS indicating desired load movement, that is, an ideal silhouette RS having a circle EN2 indicating kicking out with a thumb of a rear foot and a circle EN3 indicating landing with a heel of a front foot.

In the posture improvement assistance system SKS of the fifth embodiment, as illustrated in FIG. 23B, the output device SY teaches the pedestrian or the like HO that the load on a heel of one foot of the pedestrian or the like HO is large by performing display or the like of a current silhouette GS having a circle EN4 indicating that the load on the heel of one foot is large in comparison with the ideal silhouette RS having the circles EN2 and EN3.

As illustrated in FIG. 23C, the posture improvement assistance system SKS of the fifth embodiment teaches the pedestrian or the like HO that load on toes of both feet of the pedestrian or the like HO is large by performing display or the like of a current silhouette GS having circles EN5 and EN6 indicating that the load on the toes of both feet is large in comparison with the ideal silhouette RS having the circles EN2 and EN3.

### Effects of Fifth Embodiment

As described above, the posture improvement assistance system SKS of the fifth embodiment performs display or the like of the current silhouettes GS having the circles EN1, EN4, EN5, and EN6 indicating load situations of the pedestrian or the like HO in comparison with the ideal silhouette RS. As a result, the pedestrian or the like HO can easily understand how different the weight movement of the pedestrian or the like HO is from the ideal weight movement.

### Sixth Embodiment

### Sixth Embodiment

A posture improvement assistance system SKS of a sixth embodiment will be described.

Similarly to the posture improvement assistance system SKS of the fifth embodiment that assists improvement of weight movement of a pedestrian or the like HO, the posture improvement assistance system SKS of the sixth embodiment assists the improvement of the weight movement of the pedestrian or the like HO.

### Function, Hardware Configuration, and Software-Based Configuration of Sixth Embodiment

A function, a hardware configuration, and a software-based configuration of the posture improvement assistance system SKS of the sixth embodiment are similar to the function, the hardware configuration, and the software-based configuration of the posture improvement assistance system SKS of the first embodiment (illustrated in FIGS. 1, 13, and 14).

### Operation of Sixth Embodiment

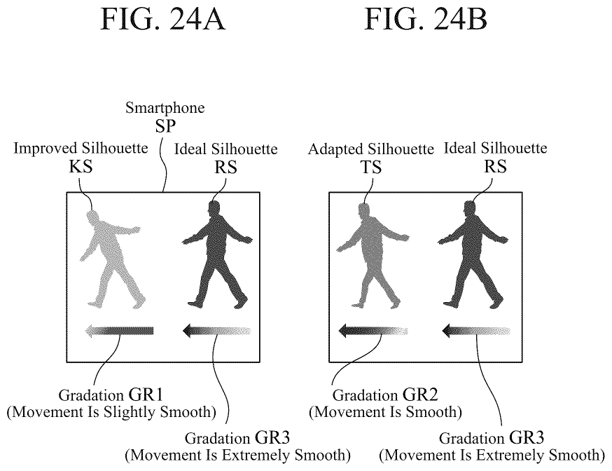

FIG. 24 illustrates an operation of the posture improvement assistance system SKS of the sixth embodiment.

The operation of the posture improvement assistance system SKS of the sixth embodiment will be described with reference to FIG. 24.

The operation of the posture improvement assistance system SKS of the sixth embodiment is basically similar to the operation of the posture improvement assistance system SKS of the first embodiment.

On the other hand, unlike the posture improvement assistance system SKS of the first embodiment, in the posture improvement assistance system SKS of the sixth embodiment, an output device SY (illustrated in FIG. 1) performs display or the like of a gradation GR in addition to display or the like of a current silhouette GS, an ideal silhouette RS, and the like.

Here, the gradation GR is one of symbols representing a current posture of the pedestrian or the like HO.

In the posture improvement assistance system SKS of the sixth embodiment, in step ST15 (illustrated in FIG. 15), as illustrated in FIG. 24A, the output device SY which is a smartphone SP teaches the pedestrian or the like HO that movement of the pedestrian or the like HO is slightly smooth by performing display or the like of an improved silhouette KS having a gradation GR1 indicating that the movement is slightly smooth in comparison with an ideal silhouette RS having a gradation GR3 indicating that the movement is extremely smooth.

In the posture improvement assistance system SKS of the sixth embodiment, in the step ST15, as illustrated in FIG. 24, the output device SY teaches the pedestrian or the like HO that movement of the pedestrian or the like HO is adapted to ideal movement by performing display or the like of an adapted silhouette TS having a gradation GR2 indicating that movement is smooth in comparison with the ideal silhouette RS having the gradation GR3 indicating that the movement is extremely smooth.

### Effects of Sixth Embodiment

As described above, in the posture improvement assistance system SKS of the sixth embodiment, in the step ST15, as illustrated in FIGS. 24A and 24B, the output device SY performs display or the like of the improved silhouette KS and the adapted silhouette TS respectively having the gradations GR1 and GR2 indicating a state of movement of the pedestrian or the like HO in comparison with the ideal silhouette RS having the gradation GR3 indicating that the movement is extremely smooth. As a result, the pedestrian or the like HO can easily understand how different the movement of the pedestrian or the like HO is from the ideal movement, in other words, can easily understand how close the movement of the pedestrian or the like HO is to the ideal movement.

### Seventh Embodiment

### Seventh Embodiment

A posture improvement assistance system SKS of a seventh embodiment will be described.

The posture improvement assistance system SKS of the seventh embodiment is different from the posture improvement assistance system SKS of the first embodiment that assists improvement of a standing state and a walking motion of a pedestrian or the like HO, and assists improvement of a walking posture of the pedestrian or the like HO.

### Function, Hardware Configuration, and Software-Based Configuration of Seventh Embodiment

A function, a hardware configuration, and a software-based configuration of the posture improvement assistance system SKS of the seventh embodiment are similar to the function, the hardware configuration, and the software-based configuration of the posture improvement assistance system SKS of the first embodiment (illustrated in FIGS. 1, 13, and 14).

### Operation of Seventh Embodiment

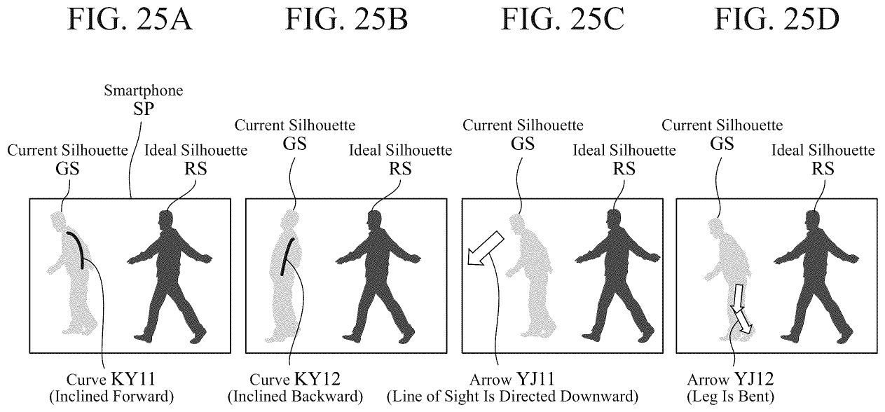

FIG. 25 illustrates an operation of the posture improvement assistance system SKS of the seventh embodiment.

The operation of the posture improvement assistance system SKS of the seventh embodiment will be described with reference to FIG. 25.

The operation of the posture improvement assistance system SKS of the seventh embodiment is basically similar to the operation of the posture improvement assistance system SKS of the first embodiment.

On the other hand, unlike the posture improvement assistance system SKS of the first embodiment, in the posture improvement assistance system SKS of the seventh embodiment, an output device SY (illustrated in FIG. 1) performs display or the like of a curve KY or an arrow YJ indicating a situation of a posture of the pedestrian or the like HO in addition to display or the like of a current silhouette GS, an ideal silhouette RS, and the like.

Here, each of the curve KY and the arrow YJ is one of symbols representing a current posture of the pedestrian or the like HO.

In step ST15 (illustrated in FIG. 15), as illustrated in FIG. 25A, the output device SY teaches the pedestrian or the like HO that the pedestrian or the like HO inclines forward by performing display or the like of a current silhouette GS having a curve KY11 indicating that the pedestrian or the like HO inclines forward in comparison with an ideal silhouette RS indicating an ideal posture of walking.

In the posture improvement assistance system SKS of the seventh embodiment, in the step ST15, as illustrated in FIG. 25B, the output device SY teaches the pedestrian or the like HO that the pedestrian or the like HO inclines backward by performing display or the like of a current silhouette GS having a curve KY12 indicating that the pedestrian or the like HO inclines backward in comparison with the ideal silhouette RS indicating an ideal posture of walking.

In the posture improvement assistance system SKS of the seventh embodiment, in the step ST15, as illustrated in FIG. 25C, the output device SY teaches the pedestrian or the like HO that the line of sight of the pedestrian or the like HO is directed downward by performing display or the like of a current silhouette GS having an arrow YJ11 indicating that the line of sight of the pedestrian or the like HO is directed downward in comparison with the ideal silhouette RS indicating an ideal posture of walking.

In the posture improvement assistance system SKS of the seventh embodiment, in the step ST15, as illustrated in FIG. 25D, the output device SY teaches the pedestrian or the like HO that a leg of the pedestrian or the like HO is bent by performing display or the like of a current silhouette GS having an arrow YJ12 indicating that the leg is bent in comparison with the ideal silhouette RS indicating an ideal posture of walking.

### Effects of Seventh Embodiment

As described above, in the posture improvement assistance system SKS of the seventh embodiment, the output device SY performs display or the like of the current silhouette GS having the curve KY11 indicating that the pedestrian or the like HO inclines forward, the curve KY12 indicating that the pedestrian or the like HO inclines backward, the arrow YJ11 indicating that a line of sight of the pedestrian or the like HO is directed downward, or the arrow YJ12 indicating that a leg of the pedestrian or the like HO is bent in comparison with the ideal silhouette RS indicating an ideal posture of walking. As a result, the pedestrian or the like HO can easily understand how different the walking posture of the pedestrian or the like HO is from the ideal posture.

### Eighth Embodiment

### Eighth Embodiment

A posture improvement assistance system SKS of an eighth embodiment will be described.

The posture improvement assistance system SKS of the eighth embodiment is different from the posture improvement assistance system SKS of the first embodiment that assists improvement of a standing state and a walking motion of a pedestrian or the like HO, and assists improvement of a walking posture of the pedestrian or the like HO similarly to the posture improvement assistance system SKS of the seventh embodiment.

### Function, Hardware Configuration, and Software-Based Configuration of Eighth Embodiment

A function, a hardware configuration, and a software-based configuration of the posture improvement assistance system SKS of the eighth embodiment are similar to the function, the hardware configuration, and the software-based configuration of the posture improvement assistance system SKS of the first embodiment (illustrated in FIGS. 1, 13, and 14).

### Operation of Eighth Embodiment

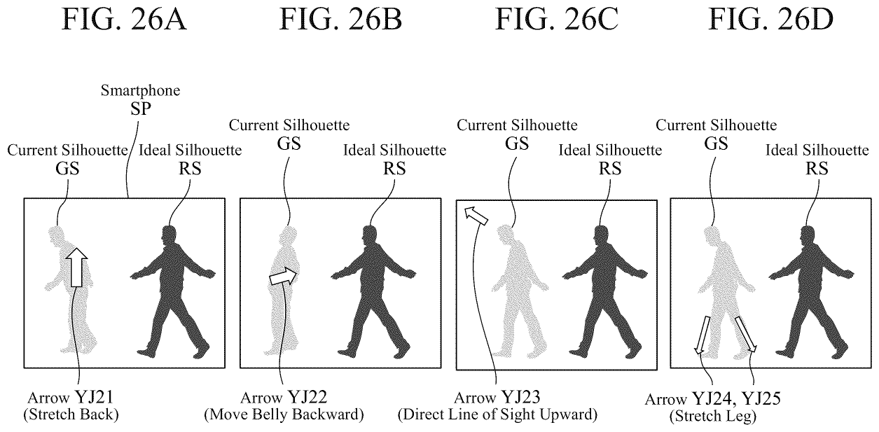

FIG. 26 illustrates an operation of the posture improvement assistance system SKS of the eighth embodiment.

The operation of the posture improvement assistance system SKS of the eighth embodiment will be described with reference to FIG. 26.

The operation of the posture improvement assistance system SKS of the eighth embodiment is basically similar to the operation of the posture improvement assistance system SKS of the first embodiment.

On the other hand, unlike the posture improvement assistance system SKS of the first embodiment, in the posture improvement assistance system SKS of the eighth embodiment, an output device SY (illustrated in FIG. 1) performs display or the like of an arrow YJ in addition to display or the like of a current silhouette GS, an ideal silhouette RS, and the like.

Here, the arrow YJ is one of symbols representing an action for improving a current posture of the pedestrian or the like HO.

In the posture improvement assistance system SKS of the eighth embodiment, in step ST15 (illustrated in FIG. 15), as illustrated in FIG. 26A, the output device SY teaches the pedestrian or the like HO that the back of the pedestrian or the like HO is desirably stretched by performing display or the like of a current silhouette GS having an arrow YJ21 indicating that the back of the pedestrian or the like HO is desirably stretched in comparison with an ideal silhouette RS indicating an ideal posture of walking.

In the posture improvement assistance system SKS of the eighth embodiment, in the step ST15, as illustrated in FIG. 26B, the output device SY teaches the pedestrian or the like HO that the belly of the pedestrian or the like HO is desirably moved backward by performing display or the like of a current silhouette GS having an arrow YJ22 indicating that the belly of the pedestrian or the like HO is desirably moved backward in comparison with the ideal silhouette RS indicating an ideal posture of walking.

In the posture improvement assistance system SKS of the eighth embodiment, in the step ST15, as illustrated in FIG. 26C, the output device SY teaches the pedestrian or the like HO that the line of sight of the pedestrian or the like HO is desirably directed upward by performing display or the like of a current silhouette GS having an arrow YJ23 indicating that the line of sight of the pedestrian or the like HO is desirably directed upward in comparison with the ideal silhouette RS indicating an ideal posture of walking.

In the posture improvement assistance system SKS of the eighth embodiment, in the step ST15, as illustrated in FIG. 26D, the output device SY teaches the pedestrian or the like HO that a leg of the pedestrian or the like HO is desirably stretched by performing display or the like of a current silhouette GS having an arrow YJ24 indicating that the leg of the pedestrian or the like HO is desirably stretched in comparison with the ideal silhouette RS indicating an ideal posture of walking.

### Effects of Eighth Embodiment

As described above, in the posture improvement assistance system SKS of the eighth embodiment, in the step ST15, the output device SY performs display or the like of the current silhouettes GS (illustrated in FIGS. 26A to 26D) having the arrow YJ21 indicating that the back of the pedestrian or the like HO is desirably stretched, the arrow YJ22 indicating that the belly of the pedestrian or the like HO is desirably moved backward, the arrow YJ23 indicating that the line of sight of the pedestrian or the like HO is desirably directed upward, or the arrow YJ24 indicating that a leg of the pedestrian or the like HO is desirably stretched in comparison with the ideal silhouette RS (illustrated in FIGS. 26A to 26D) indicating an ideal posture of walking. As a result, the pedestrian or the like HO can easily understand how to improve the posture of walking of the pedestrian or the like HO.

### Ninth Embodiment

### Ninth Embodiment

A posture improvement assistance system SKS of a ninth embodiment will be described.

The posture improvement assistance system SKS of the ninth embodiment is different from the posture improvement assistance system SKS of the first embodiment that assists improvement of a standing state and a walking motion of a pedestrian or the like HO, and assists improvement of a foot gap of the pedestrian or the like HO.

### Function, Hardware Configuration, and Software-Based Configuration of Ninth Embodiment

A function, a hardware configuration, and a software-based configuration of the posture improvement assistance system SKS of the ninth embodiment are similar to the function, the hardware configuration, and the software-based configuration of the posture improvement assistance system SKS of the first embodiment (illustrated in FIGS. 1, 13, and 14).

### Operation of Ninth Embodiment

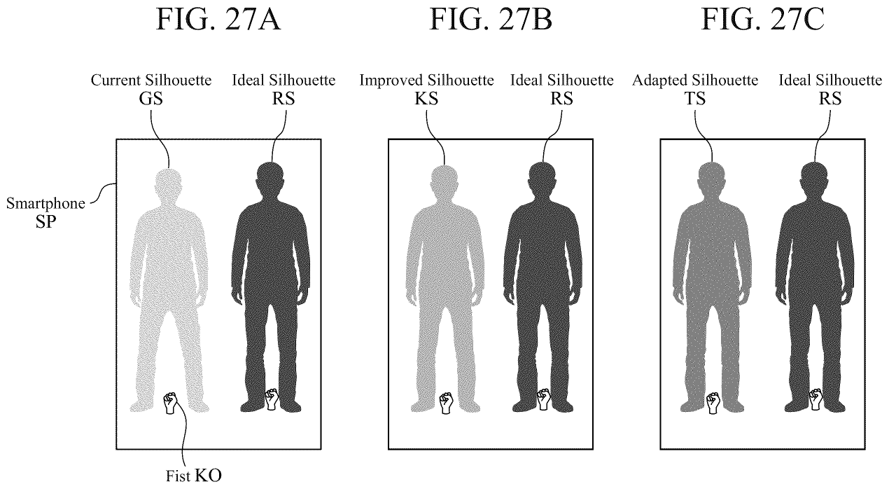

FIG. 27 illustrates an operation of the posture improvement assistance system SKS of the ninth embodiment.

The operation of the posture improvement assistance system SKS of the ninth embodiment will be described with reference to FIG. 27.

The operation of the posture improvement assistance system SKS of the ninth embodiment is basically similar to the operation of the posture improvement assistance system SKS of the first embodiment.

On the other hand, unlike the posture improvement assistance system SKS of the first embodiment, in the posture improvement assistance system SKS of the ninth embodiment, an output device SY (illustrated in FIG. 1) performs display or the like of a fist KO in addition to display or the like of a current silhouette GS, an ideal silhouette RS, and the like.

Here, the first KO is one of symbols representing a current posture of the pedestrian or the like HO.

In the posture improvement assistance system SKS of the ninth embodiment, on the other hand, in step ST15 (illustrated in FIG. 15), as illustrated in FIG. 27A, the output device SY teaches the pedestrian or the like HO that a foot gap of the pedestrian or the like HO is considerably longer than one first KO by performing display or the like of a current silhouette GS having a foot gap considerably longer than one first KO in comparison with an ideal silhouette RS having an ideal foot gap of one fist KO.

In the posture improvement assistance system SKS of the ninth embodiment, in the step ST15, as illustrated in FIG. 27B, the output device SY teaches the pedestrian or the like HO that a foot gap of the pedestrian or the like HO is slightly longer than one fist KO by performing display or the like of an improved silhouette KS having a foot gap slightly longer than one fist KO in comparison with the ideal silhouette RS having an ideal foot gap of one fist KO.

In the posture improvement assistance system SKS of the ninth embodiment, in the step ST15, the output device SY teaches the pedestrian or the like HO that a foot gap of the pedestrian or the like HO is substantially the same as one fist KO by performing display or the like of an adapted silhouette TS having a foot gap substantially the same as one fist KO in comparison with the ideal silhouette RS having an ideal foot gap of one fist KO.

### Effects of Ninth Embodiment

In the posture improvement assistance system SKS of the ninth embodiment, in the step ST15, the output device SY performs display or the like of a current silhouette GS having a foot gap considerably longer than one fist KO, an improved silhouette KS having a foot gap slightly longer than one fist KO, or an adapted silhouette TS having a foot gap substantially the same as one fist KO (illustrated in FIGS. 27A to 27C) in comparison with the ideal silhouette RS having an ideal foot gap of one fist KO (illustrated in FIGS. 27A to 27C). As a result, the pedestrian or the like HO can easily understand how different the foot gap of the pedestrian or the like HO is from the ideal foot gap.

### Modifications.

### <Modification 1>

Modification 1 of the posture improvement assistance systems SKS of the embodiments will be described.

A posture improvement assistance system SKS of Modification 1 is different from the posture improvement assistance system SKS of the first embodiment that performs display or the like of a current silhouette GS, an ideal silhouette RS, and the like separately from each other as illustrated in FIG. 17, and displays the current silhouette GS, the ideal silhouette RS, and the like in a mutually overlapping manner.

### <Function, Hardware Configuration, and Software-Based Configuration of Modification 1>

A function, a hardware configuration, and a software-based configuration of the posture improvement assistance system SKS of Modification 1 are similar to the function, the hardware configuration, and the software-based configuration of the posture improvement assistance system SKS of the first embodiment (illustrated in FIGS. 1, 13, and 14).

### <Operation of Modification 1>

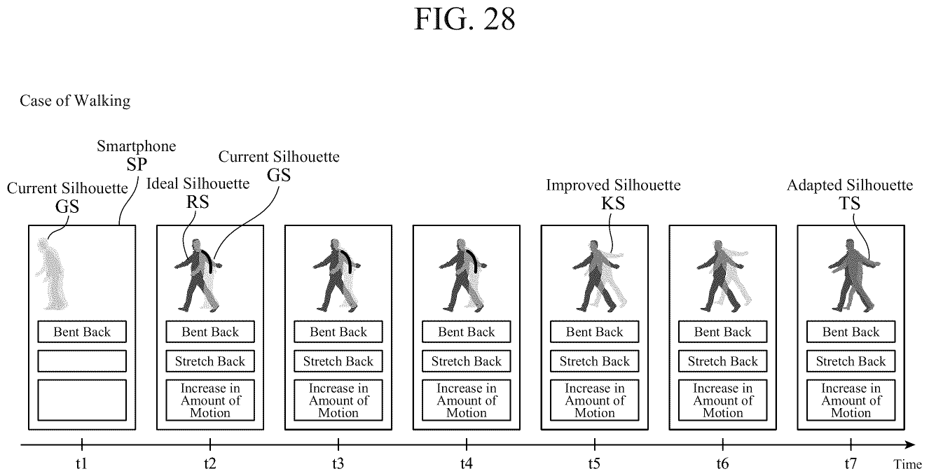

FIG. 28 illustrates an operation of the posture improvement assistance system SKS of Modification 1.

The operation of the posture improvement assistance system SKS of Modification 1 will be described with reference to FIG. 28.

The operation of the posture improvement assistance system SKS of Modification 1 is basically similar to the operation of the posture improvement assistance system SKS of the first embodiment.

On the other hand, in the posture improvement assistance system SKS of Modification 1, unlike the posture improvement assistance system SKS of the first embodiment, in a case where a pedestrian or the like HO is walking in step ST15 (illustrated in FIG. 15), as illustrated in FIG. 28, an output device SY (illustrated in FIG. 1) displays a current silhouette GS, an ideal silhouette RS, and the like of the pedestrian or the like HO in time series similarly to the illustration in FIG. 17 of the first embodiment, and performs display or the like of the current silhouette GS, the ideal silhouette RS, and the like in a mutually overlapping manner instead of performing display or the like of the current silhouette GS, the ideal silhouette RS, and the like separately from each other, unlike the illustration in FIG. 17 of the first embodiment.

As described above, in the posture improvement assistance system SKS of Modification 1, similarly to the posture improvement assistance system SKS of the first embodiment, the pedestrian or the like HO compares a posture indicated by the ideal silhouette RS with a current posture, an improved posture, and an adapted posture of the pedestrian or the like HO indicated by the current silhouette GS, the improved silhouette KS, and the adapted silhouette TS of which display or the like is performed so as to overlap the ideal silhouette RS. As a result, similarly to the posture improvement assistance system SKS of the first embodiment, the pedestrian or the like HO can easily understand how the posture of the pedestrian or the like HO is bad, how the posture of the pedestrian or the like HO is improved, and how the posture of the pedestrian or the like HO is adapted as compared with related art, and as a result, walking of the pedestrian or the like HO can be easily improved as compared with related art.

### <Modification 2>

Modification 2 of the posture improvement assistance systems SKS of the embodiments will be described.

The posture improvement assistance system SKS of Modification 2 is different from the posture improvement assistance system SKS of the first embodiment that performs display or the like of a current silhouette GS, an ideal silhouette RS, and the like separately from each other as illustrated in FIG. 18, and displays the current silhouette GS, the ideal silhouette RS, and the like in a mutually overlapping manner.

### <Function, Hardware Configuration, and Software-Based Configuration of Modification 2>

A function, a hardware configuration, and a software-based configuration of the posture improvement assistance system SKS of Modification 2 are similar to the function, the hardware configuration, and the software-based configuration of the posture improvement assistance system SKS of the first embodiment (illustrated in FIGS. 1, 13, and 14).

### <Operation of Modification 2>

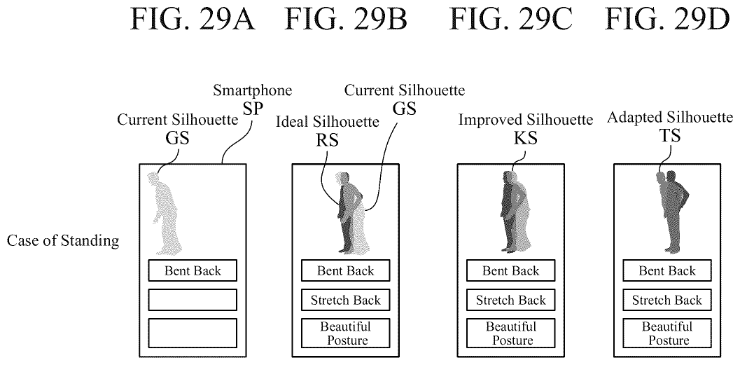

FIG. 29 illustrates an operation of the posture improvement assistance system SKS of Modification 2.

The operation of the posture improvement assistance system SKS of Modification 2 will be described with reference to FIG. 29.

The operation of the posture improvement assistance system SKS of Modification 2 is basically similar to the operation of the posture improvement assistance system SKS of the first embodiment.

On the other hand, in the posture improvement assistance system SKS of Modification 2, in a case where a pedestrian or the like HO is standing in step ST15 (illustrated in FIG. 15), as illustrated in FIGS. 29A to 29D, an output device SY (illustrated in FIG. 1) displays a current silhouette GS, an ideal silhouette RS, and the like of the pedestrian or the like HO in a snapshot manner similarly to the illustration in FIG. 18 of the first embodiment, and performs display or the like of the current silhouette GS, the ideal silhouette RS, and the like in a mutually overlapping manner instead of performing display or the like of the current silhouette GS, the ideal silhouette RS, and the like separately from each other, unlike the illustration in FIG. 18 of the first embodiment.

As described above, also in the posture improvement assistance system SKS of Modification 2, similarly to the posture improvement assistance system SKS of the first embodiment, the pedestrian or the like HO compares a posture indicated by an ideal silhouette RS with a current posture, an improved posture, and an adapted posture of the pedestrian or the like HO indicated by a current silhouette GS, an improved silhouette KS, and an adapted silhouette TS of which display or the like is performed so as to overlap the ideal silhouette RS. As a result, similarly to the posture improvement assistance system SKS of the first embodiment, the pedestrian or the like HO can easily understand how the posture of the pedestrian or the like HO is bad, how the posture of the pedestrian or the like HO is improved, and how the posture of the pedestrian or the like HO is adapted as compared with related art, and as a result, a standing position of the pedestrian or the like HO can be easily improved as compared with related art.

The embodiments may be combined, and the components of the embodiments may be appropriately deleted or changed, or other components may be added without departing from the gist of the present disclosure.

### INDUSTRIAL APPLICABILITY

The posture improvement assistance system according to the present disclosure can be used to enable easier improvement of a posture than related art.

### REFERENCE SIGNS LIST

DA: alternative, DO: motivation, EN: circle, GE: current posture, GR: gradation, GS: current silhouette, GZ: image, HO: pedestrian or the like, KA: camera, KM: basic model, KO: fist, KP: portable projector, KR: detection amount, KS: improved silhouette, KT: shoe, NY: input device, RS: ideal silhouette, SE: sensor, SG: smart glasses, SH: processing device, SKS: posture improvement assistance system, SP: smartphone, SW: smartwatch, SY: output device, TA: tablet, TP: attachment type projector, TS: adapted silhouette, YJ: arrow

1. A posture improvement assistance system comprising:
processing circuitry configured to
receive input of user information which is information regarding a posture of a user;
generate a current silhouette representing a current posture of the user on a basis of the user information; and
output the current silhouette and an ideal silhouette representing an ideal posture of the user.

2. The posture improvement assistance system according to claim 1, wherein
the processing circuitry extracts an ideal silhouette corresponding to the current silhouette of the user by searching a database in which an image of the user, a detection amount of the user, the current silhouette, and the ideal silhouette are associated with each other on a basis of at least one or more of the image of the user, the detection amount of the user, and the current silhouette of the user which are the user information.

3. The posture improvement assistance system according to claim 1, wherein
the processing circuitry additionally outputs at least one of a current posture of the user, an action that the user should take in order to improve the posture of the user, and a motivation for the user to perform a task.

4. The posture improvement assistance system according to claim 1, wherein
the processing circuitry outputs, instead of the current silhouette, at least one of an improved silhouette indicating a posture improved from the current posture of the user and an adapted silhouette from the improved posture of the user to a posture adapted to an ideal posture represented by the ideal silhouette.

5. The posture improvement assistance system according to claim 1, wherein
the processing circuitry outputs at least one of the current silhouette, an improved silhouette, and an adapted silhouette which partially or entirely represent a posture of the user, and an ideal silhouette partially or entirely representing an ideal posture of the user.

6. The posture improvement assistance system according to claim 1, wherein
output of at least one of the current silhouette, an improved silhouette, and an adapted silhouette partially representing a posture of the user, and an ideal silhouette which partially represent an ideal posture of the user, and output of at least one of the current silhouette, the improved silhouette, and the adapted silhouette which entirely represent the posture of the user, and the ideal silhouette entirely representing the ideal posture of the user are performed in a switched manner or simultaneously.

7. The posture improvement assistance system according to claim 1, wherein
the processing circuitry outputs a symbol indicating at least one of the current posture of the user and an action for improving the current posture of the user.

8. The posture improvement assistance system according to claim 1, wherein
the processing circuitry generates a real silhouette representing the user from an image of the user which is the user information, and generates the current silhouette by correcting the generated real silhouette by referring to a basic model which is a standard posture of a human prepared in advance.

9. The posture improvement assistance system according to claim 4, wherein
the processing circuitry outputs one of the current silhouette, the improved silhouette, and the adapted silhouette and the ideal silhouette separately from each other.

10. The posture improvement assistance system according to claim 4, wherein
the processing circuitry outputs one of the current silhouette, the improved silhouette, and the adapted silhouette and the ideal silhouette in a mutually overlapping manner.

11. A processing device comprising:
processing circuitry configured to
acquire user information which is information regarding a posture of a user; and
generate a current silhouette representing a current posture of the user on a basis of the user information.

12. A posture improvement assistance method comprising:
receiving input of user information which is information regarding a posture of a user;
generating a current silhouette representing a current posture of the user on a basis of the user information; and
outputting the current silhouette and an ideal silhouette representing an ideal posture of the user.

13. A processing method comprising:
acquiring user information which is information regarding a posture of a user; and
generating a current silhouette representing a current posture of the user on a basis of the user information.
